(19) United States
(12) Patent Application Publication
(10) Pub. No.: US 2024/0157272 A1
(43) Pub. Date: May 16, 2024

(54) A METHOD FOR PURIFYING A FEED IN A CHROMATOGRAPHY SYSTEM

(71) Applicant: Puridify Limited, Stevenage, Hertfordshire (GB)

(72) Inventors: Gunnar Malmquist, Uppsala (SE); Niklas Jungnelius, Uppsala (SE)

(21) Appl. No.: 18/561,252

(22) PCT Filed: Jun. 13, 2022
(86) PCT No.: PCT/EP2022/066012

§ 371 Date: Nov. 15, 2023

(30) Foreign Application Priority Data
Jun. 23, 2021 (GB) 2109015.4

(51) Int. Cl. B01D 15/18; B01D 15/20; C12M 1/00

(52) U.S. Cl. CPC B01D 15/1885; B01D 15/203; C12M 47/12

(57) ABSTRACT

The present invention relates to a method for purifying a feed comprising a concentration of a target product in a chromatography system (10) having a first adsorption purifying unit (13). The first adsorption purifying unit has a capacity for binding the target product and is configured to receive the feed from a first holding tank (12), receiving continuous feed from a bioreactor (11), and to provide the target product (14) at an outlet (13b). The method comprises: a) loading (S20) the first adsorption purifying unit (13) with a volume of feed provided from the first holding tank (12), the volume of feed comprising an amount of the at least one target product corresponding to less than, or equal to, the capacity for binding the target product in the first adsorption purifying unit, b) washing, eluting, cleaning and regenerating (S30) the first adsorption purifying unit (13) while filling the first holding tank (12) with feed, said first holding tank (12) having a volume of at least the amount of the feed provided by the bioreactor (11) during this step, and repeating step a) and b) for a predetermined number of cycles.

### TECHNICAL FIELD

The present disclosure relates to method for purifying a feed comprising a target product in a chromatography system, especially a chromatography system with a short cycle time. The present disclosure also relates to a chromatography system and computer program for carrying out the method.

### BACKGROUND

Chromatography systems are widely used to extract target products available in a feed from a bioreactor. For efficient use, large columns have traditionally been used to capture the target product, but with the drawback of time consuming procedures for washing, eluting, cleaning, CIP, and regenerating the columns before they may be loaded with feed again. In addition, it is beneficial to continuously provide feed from the bioreactor to further increase the efficiency of the purifying procedure by using multiple columns.

In continuous chromatography, several identical, or almost identical, columns are connected in an arrangement that allows columns to be operated in series and/or in parallel, depending on the method requirements. Thus, all columns can be run in principle simultaneously, but with slightly shifted method steps. The procedure can be repeated, so that each column is loaded, eluted, and regenerated several times in the process. Compared to ‘conventional’ chromatography, wherein a single chromatography cycle is based on several consecutive steps, such as: load the sample, wash, elution, strip, Clean-In-Place (CIP) and re-equilibration, before the column may be used for another batch, in continuous chromatography based on multiple identical columns all these steps may occur simultaneously but on different columns each.

However, this is a rather complex and expensive system, and it is difficult to manufacture columns that are identical. Thus, there is a desire to develop a system which is not dependent on identical columns for efficient functionality when purifying a feed to obtain a target product.

### SUMMARY

An object of the present disclosure is to provide a method which seeks to mitigate, alleviate, or eliminate one or more of the above-identified deficiencies in the art and disadvantages singly or in any combination and to provide an improved chromatography system .

This object is obtained by a method for purifying a feed comprising a concentration of at least one target product in a chromatography system having at least a first adsorption purifying unit. The at least first adsorption purifying unit has a capacity for binding the target product and is configured to receive the feed from a first holding tank which is configured to receive a continuous feed from a bioreactor. The at least first adsorption purifying unit is also configured and to provide the at least one target product at an outlet. The method comprising:
a) loading the at least first adsorption purifying unit with a volume of feed provided from the first holding tank, the volume of feed comprising an amount of the at least one target product corresponding to less than, or equal to, the capacity for binding the target product in the at least first adsorption purifying unit;
b) when step a) is completed, washing, eluting, cleaning and regenerating the at least first adsorption purifying unit while filling the first holding tank with feed, said first holding tank having a volume of at least the amount of the feed provided by the bioreactor during this step;
c) repeating step a) and b) for a predetermined number of cycles.

According to an aspect, a chromatography system for purifying a feed comprising a concentration of at least one target product is provided. The chromatography system has at least a first adsorption purifying unit having a capacity for binding the target product and is configured to receive the feed from at least a first holding tank, which is configured to receive a continuous feed from a bioreactor. The at least first adsorption purifying unit is also configured to provide the at least one target product at an outlet, wherein the chromatography system is further configured to perform the steps:
a) loading the at least first adsorption purifying unit with a volume of feed provided from the first holding tank, the volume of feed comprising an amount of the at least one target product corresponding to less than, or equal to, the capacity for binding the target product in the at least first adsorption purifying unit;
b) when step a) is completed, washing, eluting, cleaning, and regenerating the at least first adsorption purifying unit while filling the first holding tank with feed, said first holding tank having a volume of at least the amount of the feed provided by the bioreactor during this step;
c) repeating step a) and b) for a predetermined number of cycles.

An advantage with the present invention is that much simpler setup and chromatography system is achieved when a continuous flow of feed is provided from the bioreactor.

Another advantage is that a chromatography system with a small holding tank may be implemented which makes it easier to use, change and monitor during operations.

Further aspects and advantages may be obtained from the detailed description.

### BRIEF DESCRIPTION OF THE DRAWINGS

The foregoing will be apparent from the following more particular description of the example embodiments, as illustrated in the accompanying drawings in which like reference characters refer to the same parts throughout the different views. The drawings are not necessarily to scale, emphasis instead being placed upon illustrating the example embodiments.

FIG. 1a illustrates a first example embodiment of a chromatography system according to the present disclosure;

FIG. 1b illustrates an alternative example embodiment of the chromatography system in FIG. 1a;

FIG. 2 illustrates a second example embodiment of a chromatography system according to the present disclosure;

FIG. 3 illustrates a third example embodiment of a chromatography system according to the present disclosure;

FIG. 4 illustrates a fourth example embodiment of a chromatography system according to the present disclosure;

FIG. 5 illustrates a fifth example embodiment of a chromatography system according to the present disclosure;

FIG. 6 is a schematic illustration of a control unit according to the present disclosure;

FIG. 7 is a flow chart illustrating a method according to the present disclosure; and

FIGS. 8-10 are graphs illustrating three examples.

### DETAILED DESCRIPTION

Aspects of the present disclosure will be described more fully hereinafter with reference to the accompanying drawings. The apparatus and method disclosed herein can, however, be realized in many different forms and should not be construed as being limited to the aspects set forth herein. Like numbers in the drawings refer to like elements throughout.

The terminology used herein is for the purpose of describing particular aspects of the disclosure only, and is not intended to limit the invention. As used herein, the singular forms “a”, “an” and “the” are intended to include the plural forms as well, unless the context clearly indicates otherwise.

Some of the example embodiments presented herein are directed towards a chromatography system. As part of the development of the example embodiments presented herein, a problem will first be identified and discussed. Continuous feed from a bioreactor is desirable since this will increase the speed of the process, and in order to facilitate this a plurality of adsorption purifying units, such as chromatography columns or membranes, have been used to create a continuous chromatography process. However, this requires multiple adsorption purifying units with identical, or at least similar, performance and a complex chromatography system with a multitude of valves and a complex control system to manage the switching of the valves.

The current disclosure describes a chromatography system where a continuous flow from the bioreactor is maintained using only one adsorption purifying unit (or a plurality of parallel-connected units) together with a holding tank connected in series between the bioreactor and the adsorption purifying unit(s). A short cycle time will also receive additional benefits, such as the holding tank will be very small (less than 2% of the daily volumetric output of the bioreactor).

In this disclosure, each adsorption purifying unit is controlled to produce a target product and each have a maximum capacity for binding the target product. Loading is performed based on process parameters controlled by a control unit as is obvious for a skilled person in the art. For example loading may be performed until break-through occurs, or a certain percentage over the point where break-through occurred, or be performed with a safety margin with loading a volume corresponding to less than breakthrough.

The concentration of the target product in the feed will impact the volume of feed loaded into the adsorption purifying unit as well as the required size of the holding tank.

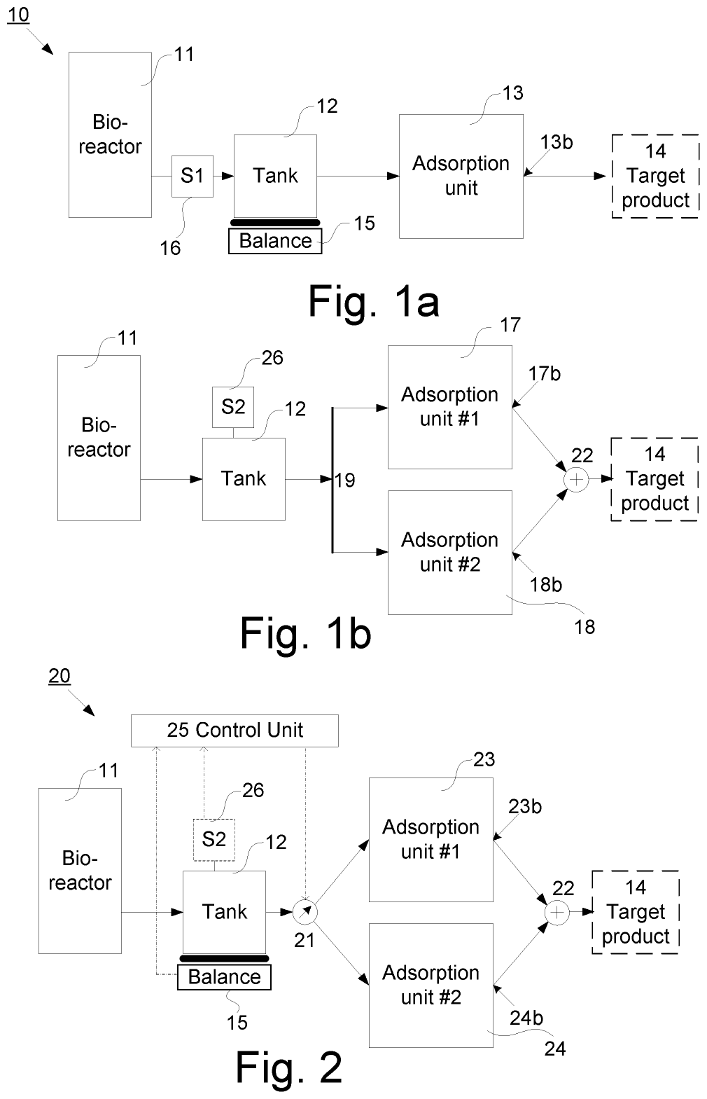

FIG. 1a is a schematic illustration of a first example embodiment of a chromatography system 10 according to the present disclosure. In addition to the features described below, a chromatography system also comprises pumps, valves, sensors, etc. to provide the desired functionality, which is obvious for a skilled person in the art and therefore will not be included in this disclosure. The chromatography system 10 comprises a bioreactor 11 providing a continuous feed comprising a concentration of at least one target product to a holding tank 12 at a feed flow rate, R_{F}, via an optional inline sensor 16 (also denoted S1) and an adsorption purifying unit 13 having a capacity for binding the target product and outputting the target product 14 through an outlet 13b. The holding tank may be provided with a balance 15 for monitoring the weight of the holding tank 12 during operations. The chromatography system also comprises a control unit (not shown) that controls the process based on certain parameters. An example of such parameter is the concentration of target product in the feed provided into the holding tank 12.

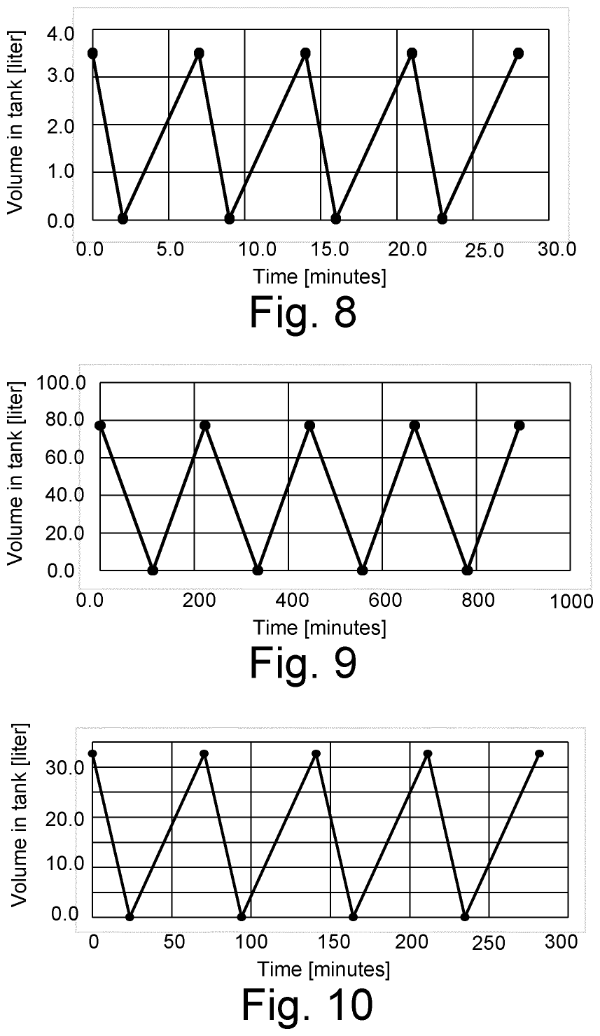

The control unit is configured to control the process, which comprises loading the adsorption purifying unit 13 with a volume of feed provided from the holding tank 12, wherein the volume corresponds to less than equal to the capacity for binding the target product in the adsorption purifying unit 13. Thus, the volume is dependent on the concentration of target product in the feed and the capacity of binding the target product in the adsorption purifying unit. When the loading is completed, the process also comprises a post-loading step comprising washing, eluting, cleaning and regenerating the adsorption purifying unit 13 while filling the holding tank 12 with feed from the bioreactor 11. The holding tank is selected to have a volume of at least the amount of the feed provided by the bioreactor 11 during the post-loading step. The size of the holding tank 12 is determined based on the feed flow rate, R_{F}, from the bioreactor, and the duration of the post-loading step. Illustrative examples are provided in connection with FIGS. 8-10.

The holding tank 12 has a maximum volume and when the loading step is completed, the holding tank optionally contains only a fraction of the total volume. The fraction of the total volume is larger than zero, but may be any portion of the maximum volume, such as ¾, ¼ of the maximum volume. Alternatively, the holding tank 12 is empty, or very close to empty, after loading. However, this introduces a risk of sucking air into the purifying unit 13 at the end of the loading step.

When the post-loading step is completed, the control unit repeats the loading and post-loading steps for a predetermined number of cycles. The predetermined number of cycles may be selected based on experience from previous runs using the same type of adsorption purifying units, or be based on the performance of the adsorption purifying unit 13. The performance may be monitored using sensors (not shown) to detect the status of the adsorption purifying unit. Status is obtained by monitoring the change in certain parameters over the adsorption purifying unit, such as pressure difference (dP), or comparing parameters before and after the purifying unit, such as UV, pH, conductivity, etc. Also, it is possible to evaluate the UV elution peak area and peak shape or salt transition shapes.

When the control unit initiates the loading step the adsorption purifying unit 13 is loaded with feed from the holding tank 12 at a loading flow rate, R_{L}. In this example, the continuous flow of feed into the holding tank 12 from the bioreactor 11 requires the feed flow rate R_{F} to be lower than the loading flow rate R_{L}, R_{F}<R_{L}. If this is not the case, the flow of feed into the holding tank 12 needs to be reduced, or stopped, in order to avoid overfilling of the holding tank 12.

The loading step of the adsorption purifying unit 13 may be performed during a time period, T, while the holding tank 12 is filled with feed. The time period, T, may be a fixed time period that is set to ensure that the amount of target product loaded into the adsorption purifying unit 12 is restricted to avoid break-through. Alternatively, the time period, T, may be dynamic to ensure that the volume of feed loaded into the adsorption purifying unit 12 is adapted to variations in the concentration of target product in the holding tank 12 based on variations in concentration of the target product in the feed from the bioreactor 11 and/or changes in performance of the adsorption purifying unit 12. A target product concentration, C_{TP}, may be determined using the optional sensor 16, which monitors the concentration in the feed from the bioreactor and the balance 15 (which may be used to determine the volume of feed in the holding tank 12 at any given instance). Alternatively, a sensor 26 may be introduced into the holding tank to measure the concentration in the holding tank as illustrated in FIG. 2. The time period, T, is thereafter set based on the target product concentration in the holding tank.

As mentioned above, a balance may be included in the chromatography system, and the balance 15 is configured to measure a weight, W, of the holding tank 12, and the loading and post-loading steps may be initiated based on the measured weight of the holding tank 12. The loading step may be initiated when the weight of the holding tank 12 is above a first weight threshold W1, i.e. W>W1, indicating that the amount of feed in the holding tank 12 is enough to ensure that the amount of target product in the feed needed to load the adsorption purifying unit 13 is available during loading. The first weight threshold W1 is also based on the feed flow rate from the bioreactor. The post-loading step may be initiated when the weight of the holding tank 12 is below a second weight threshold W2, i.e. W<W2, indicating that a small fraction of feed is available in the holding tank 12 at the end of the loading step (to avoid sucking air into the adsorption purifying unit 13).

FIG. 1b illustrates an alternative example embodiment of the chromatography system in FIG. 1a with two parallel-connected adsorption purifying units 17 and 18 provided with feed from the holding tank 12 via a manifold 19. Instead of having a balance (as in FIG. 1a), the holding tank is provided with a sensor 26 (also denoted S2) configured to measure the concentration in the holding tank 12 used to determine the target product concentration C_{TP} in the holding tank 12. An example of such a sensor is Surface Plasmon Resonance, SPR, which may be installed in the holding tank.

Furthermore, each adsorption purifying unit 17, 18 has a capacity for binding the target product and outputting the target product 14 through respective outlets 17b, 18b via a combiner 22. The loading flow rate R_{L} for each adsorption purifying unit may be lower than, or equal to the feed flow rate R_{F} as long as the combined loading flow rate of the parallel-connected adsorption purifying units 17, 18 is higher than the feed flow rate R_{F} from the bioreactor 11.

FIGS. 2, 3 and 5 illustrate example chromatography systems having at least a first and a second adsorption purifying units, where each example chromatography system is provided with valves to redirect the feed from the bioreactor 11 from the first adsorption purifying unit 23 to the second adsorption purifying unit 24 when the predetermined number of cycles (loading and post-loading steps) have been performed using the first adsorption purifying unit 23. When the feed has been redirected to the second adsorption purifying unit 24, the loading and post-loading steps are repeated for a predetermined number of cycles while providing feed from the bioreactor 11 to the second adsorption purifying unit 24.

FIG. 2 illustrates a second example embodiment of a chromatography system 20 according to the present disclosure, which is similar to the chromatography system 10 described in connection with FIG. 1. The chromatography system 20 comprises a bioreactor 11 providing continuous feed comprising a concentration of at least one target product to a holding tank 12 at a feed flow rate, R_{F}, and two adsorption purifying units 23, 24 each having a capacity for binding the target product and outputting the target product 14 through respective outlets 23b, 24b via a combiner 22. In this example embodiment, the chromatography system 20 further comprises a valve 21 having at least one inlet for receiving feed from the holding tank 12, a first outlet connected to the first adsorption purifying unit 23 and a second outlet connected to the second adsorption purifying unit 24. The holding tank 12 may be provided with a balance 15 for monitoring the weight of the holding tank 12 during operations. The chromatography system 20 also comprises a control unit 25 that controls the process based on certain parameters.

The control unit 25 is configured to redirect the feed flow from the bioreactor 11 to the second adsorption purifying unit 24 using the valve 21 after cycling the first adsorption purifying unit 23 for the predetermined number of cycles. Optionally, the chromatography system comprises a sensor 26 (also denoted S2) configured to measure the concentration in the holding tank 12 used to determine the target product concentration C_{TP} in the holding tank 12. An example of such a sensor is based in Surface Plasmon Resonance, SPR, which may be installed in the holding tank. The outlet 24b from the second adsorption purifying unit 24 may provide the target product directly or via the combiner 22.

The main advantage of a chromatography system 20 with the valve 21 and the combiner 22 is that a switch between adsorption purifying units may be achieved seamlessly without affecting the production.

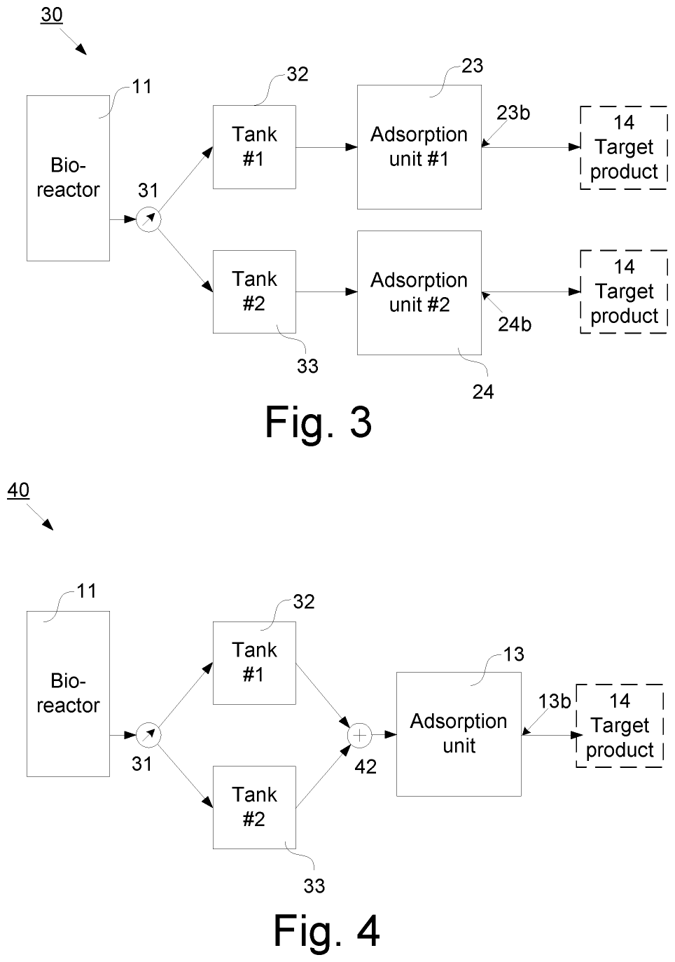

FIG. 3 illustrates a third example embodiment 30 of a chromatography system according to the present disclosure, which is similar to the chromatography system 20 described in connection with FIG. 2. The chromatography system 30 comprises a bioreactor 11 providing continuous feed comprising a concentration of at least one target product to a first or a second holding tank 32, 33 at a feed flow rate, R_{F}, via a valve 31 having an inlet for receiving feed from the bioreactor 11, a first outlet connected to a first holding tank 32 and a second outlet connected to a second holding tank 33. The system further comprises two adsorption purifying units 23, 24 each having a capacity for binding the target product and outputting the target product 14 through respective outlets 23b, 24b in separate vessels for quality control. It is of course possible to only use one vessel for the target product by combining the outputs 23b, 24b via a combiner as described in FIG. 2. In this example embodiment, each adsorption purifying unit 23, 24 respectively receives feed from a first and second holding tank 32, 33. The chromatography system 300 also comprises a control unit (not shown) that controls the process based on certain parameters.

The control unit is configured to redirect the feed flow from the bioreactor 11 to the second holding tank 33 using the valve 31 after cycling the first adsorption purifying unit 23 for the predetermined number of cycles. The outlet 24b from the second adsorption purifying unit 24 may provide the target product via a combiner 22.

Optionally, the chromatography system comprises a sensor (not shown) configured to determine the target product concentration C_{TP} in each holding tank.

Optionally, a balance (not shown) is provided to measure the weight of each holding tank.

The main advantage of a chromatography system 30 with the valve 31 and the combiner 22 is that a switch between adsorption purifying units may be achieved seamlessly at the same time as changing the holding tank without affecting the production.

FIG. 4 illustrates a fourth example embodiment of a chromatography system 40 according to the present disclosure, which is similar to the chromatography system 10 described in connection with FIG. 1. The chromatography system 40 comprises a bioreactor 11 providing continuous feed comprising a concentration of at least one target product to a first or a second holding tank 32, 33 at a feed flow rate, R_{F}, via a valve 31 having an inlet for receiving feed from the bioreactor 11, a first outlet connected to a first holding tank 32 and a second outlet connected to a second holding tank 33. The system further comprises an adsorption purifying unit 13 having a capacity for binding the target product and outputting the target product 14 through outlet 13b. In this example embodiment, the chromatography system 40 further comprises a combiner 42 combining the feed flow from the respective holding tank 32, 33 to the adsorption purifying unit 13. The chromatography system 40 also comprises a control unit (not shown) that controls the process based on certain parameters.

The control unit is configured to redirect the feed from the bioreactor 11 from the first holding tank 32 to a second holding tank 33; and load the first adsorption purifying unit 13 with feed from the second holding tank 33.

Optionally, the chromatography system comprises a sensor (not shown) configured to determine the target product concentration C_{TP} in each holding tank.

Optionally, a balance (not shown) is provided to measure the weight of each holding tank.

The main advantage of a chromatography system 40 with the valve 31 and the combiner 42 is that a switch between holding tanks may be achieved seamlessly without affecting the production.

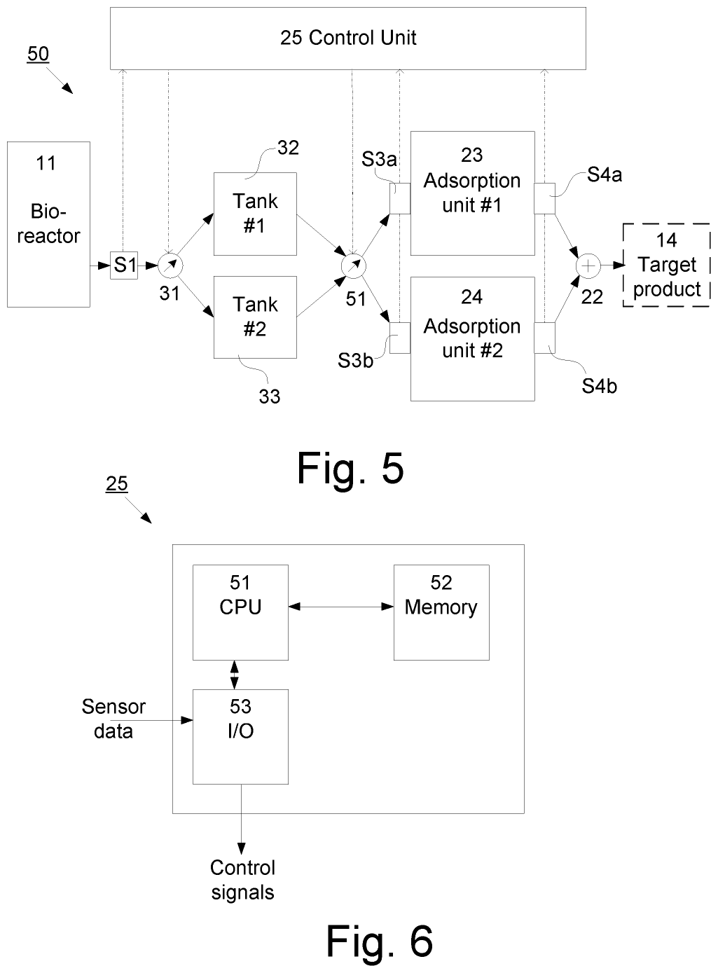

FIG. 5 illustrates a fifth example embodiment of a chromatography system 50 according to the present disclosure, which is a combination of the example embodiments disclosed in connection with FIGS. 2 and 4. In order to be able to combine these example embodiments, a valve 51 is included having a first inlet for receiving feed from the first holding tank 32, a second inlet for receiving feed from the second holding tank 33, a first outlet connected to the first adsorption purifying unit 23 and a second outlet connected to the second adsorption purifying unit 24. Furthermore, each adsorption purifying units 23 and 24 are provided with inlet sensors S3a, S3b and outlet sensors S4a, S4b to monitor the status of the adsorption purifying unit currently in operation. Status may be obtained by monitoring the change in certain parameters over the adsorption purifying unit using the inlet and outlet sensors, such as pressure difference (dP), or comparing parameters before and after the purifying unit, such as UV, pH, conductivity, etc. Also, it is possible to evaluate the UV elution peak area and peak shape or salt transition shapes.

The control unit (25) is configured to redirect the feed flow from the bioreactor 11 to the at least second adsorption purifying unit 24 using the second valve 51 after the predetermined number of cycles.

FIG. 6 is a schematic illustration of a control unit 25 (denoted CPU) according to the present disclosure comprising a processor 51 and a memory 52 (which is accessible to the processor 51) and input/output circuitry (denoted I/O) configured to receive sensor data and control the process via control signals.

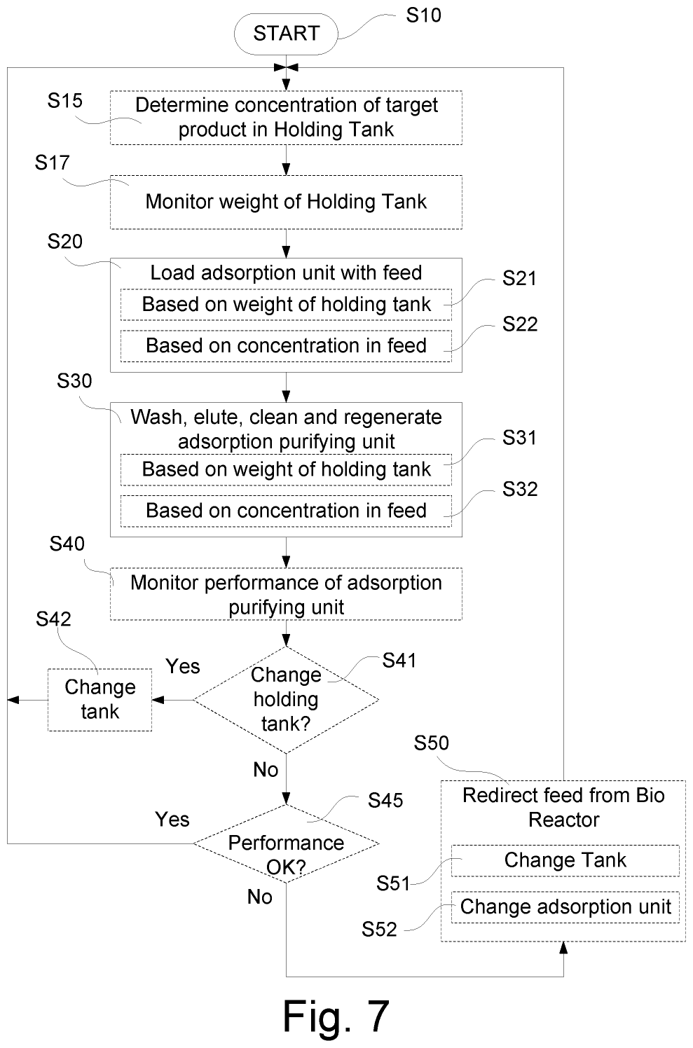

FIG. 7 is a flow chart illustrating a method for purifying a feed comprising a concentration of at least one target product in a chromatography system having at least a first adsorption purifying unit, the at least first adsorption purifying unit has a capacity for binding the target product and is configured to receive the feed from a first holding tank and to provide the at least one target product at an outlet, wherein the first holding tank is configured to receive continuous feed from a bioreactor.

The process starts in step S10 and there are a few optional steps, wherein the first optional step S15 is determining concentration of target product in holding tank and the second optional step S17 is to monitor the weight of the holding tank. These steps are described in more detail below. Loading S20 the first adsorption purifying unit with feed from the bioreactor is the next step, in which the at least first adsorption purifying unit is loaded with a volume of feed provided from the first holding tank, the volume of feed comprising an amount of the at least one target product corresponding to less than, or equal to, the capacity for binding the target product in the at least first adsorption purifying unit. The loading is performed during a time period, T, while supplying feed from the bioreactor to the first holding tank.

Optionally, the chromatography system further comprises a weight sensor configured to measure, in step S17, a weight, W, of the first holding tank, wherein loading is initiated based on the measured weight of the first holding tank. As an example, the loading may be initiated S21 when the measured weight W of the first holding tank is above a first weight threshold W1, W>W1;

Optionally, the chromatography system further comprises a sensor to determine, in step S15, the concentration of target product in holding tank, wherein loading is initiated S22 based on the target product concentration, C_{TP}, in the first holding tank.

When loading S20 is completed, the process continues with a post-loading step comprising washing, eluting, cleaning and regenerating S30 the at least first adsorption purifying unit while filling the first holding tank with feed The first holding tank has a volume of at least the amount of the feed provided by the bioreactor during this step. The first holding tank has a maximum volume and may contain a fraction of the total volume when loading is completed.

Optionally, the post-loading step is initiated S31 when the measured weight W of the first holding tank is below a second weight threshold W2, W<W2.

Optionally, the post-loading step is initiated S32 based on the target product concentration, C_{TP}, in the first holding tank.

Before repeating steps for loading S20 and post-loading S30, for a predetermined number of cycles, the process may comprise a number of optional steps.

The performance of the first adsorption purifying unit may be monitored in step S40. If the performance is OK, step S45, another cycle is initiated with the same adsorption purifying unit. If the performance is not OK, the process continues to step S50 where the feed flow is redirected from the first adsorption purifying unit to a second adsorption purifying unit. This may also include changing the holding tank S51 or only the adsorption purifying unit is changed S52 before initiating loading of the second adsorption purifying unit.

Optionally, there is a decision to only change the holding tank, step S41. This may be caused by a contamination of the holding tank or to replace the holding tank for another reason, e.g. change volume of holding tank. If so the holding tank is changed S42 and the process continues with loading S20 feed into the adsorption purifying unit currently in operations.

The loading may be performed at a loading flow rate, R_{L}, and the feed from the bioreactor may be introduced into the first holding tank at a feed flow rate, R_{F}, wherein the feed flow rate is lower than the loading flow rate, R_{F}<R_{L}.

The predetermined number of cycles may be based on the performance of the at least first adsorption purifying unit.

FIGS. 8-10 are graphs illustrating three examples. In all examples, a bioreactor with the following specification is used: Volume of bioreactor 1000 litres outputting a feed having a perfusion rate of 1000 litres/24 h and perfusate flow of 0.69 litre/min. The titer is 3 g/litre and the mass output is 3000 g/24 h. The total possible load volume per cycle has been calculated based on the titer 3 g/litre and a load of 24 mg/ml.

FIG. 8 shows a first example illustrating a change in volume over time within a holding bag during cycling of a small adsorption purifying unit having the following specifications:

The adsorption purifying unit is a membrane purifying unit with a volume of 0.608 litre, a loading flow of 4 Membrane Volume/min and a total possible load volume per cycle of 4.86 litres. The downstream process, DSP, flow is 2.43 litres/min. The bioreactor will produce 3.47 litres of feed during the non-loading step (comprising washing, eluting, cleaning and regenerating the adsorption purifying unit). The loading step is 2 minutes and the total cycle time is 7 minutes, which yields 206 possible cycles/24 hours. Thus, based on these conditions it is possible to calculate the minimum volume of the holding tank: 3.47 litres, which corresponds to 0.35% of the total volume of the bioreactor and 571% of the adsorption purifying unit.

As may be seen in FIG. 8, the amount of feed in the holding tank is 3.5 litres at t=0 min, and during loading the volume in the holding tank is loaded into the adsorption purifying unit together with additional feed provided from the bioreactor. When loading is completed at t=2 min, the amount of feed in the holding tank is in this example 0.028 litre before the holding tank is filled again during the non-loading step until t=7 min.

FIG. 9 shows a second example illustrating a change in volume over time within a holding bag during cycling of a column as adsorption purifying unit having the following specifications:

The column has a volume of 8.296 litres, a loading flow of 0.166667 Column Volume/min and a total possible load volume per cycle of 154.86 litres. The DSP flow is 1.38 litres/min. The bioreactor will produce 77.08 litres of feed during the non-loading step (comprising washing, eluting, cleaning and regenerating the column). The loading step is 112 minutes and the total cycle time is 223 minutes, which yields 6 possible cycles/24 hours. Thus, based on these conditions it is possible to calculate the minimum volume of the holding tank: 77.08 litres, which corresponds to 7.71% of the total volume of the bioreactor and 929% of the column. This requires a rather large holding tank to be able to have a bioreactor with a continuous flow of feed.

As may be seen in FIG. 9, the amount of feed in the holding tank is 77.1 litres at t=0 min, and during loading the volume in the holding tank is loaded into the column together with additional feed provided from the bioreactor. When loading is completed at t=112 min, the amount of feed in the holding tank is in this example 0.017 litre before the holding tank is filled again during the non-loading step until t=223 min.

FIG. 10 shows a third example illustrating a change in volume over time within a holding bag during cycling of a column as adsorption purifying unit adapted for rapid cycling having the following specifications:

The column has a volume of 4.171 litres, a loading flow of 0.5 Column Volume/min and a total possible load volume per cycle of 48.94 litres. The DSP flow is 2.09 litres/min. The bioreactor will produce 32.64 litres of feed during the non-loading step (comprising washing, eluting, cleaning and regenerating the column). The loading step is 23.47 minutes and the total cycle time is 70.47 minutes, which yields 20 possible cycles/24 hours. Thus, based on these conditions it is possible to calculate the minimum volume of the holding tank: 32.64 litres, which corresponds to 3.26% of the total volume of the bioreactor and 783% of the column. Although the required volume of the holding tank is reduced by more than 50% compared to the second example in FIG. 9, the holding tank is still large when having a continuous flow of feed provided by the bioreactor.

As may be seen in FIG. 10, the amount of feed in the holding tank is 32.7 litres at t=0 min, and during loading the volume in the holding tank is loaded into the column together with additional feed provided from the bioreactor. When loading is completed at t=23.47 min, the amount of feed in the holding tank is in this example 0.061 litre before the holding tank is filled again during the non-loading step until t=70.47 min.

It should be noted that it is desirable to avoid completely emptying the holding tank during loading since air might be sucked into the adsorption purifying unit. An amount corresponding to 10% of the maximum volume of the holding tank, e.g. 0.4 litre in the first example, may be left in the holding tank at the end of the loading step before the holding tank is refilled with feed during the non-loading step.

The major benefit with the chromatography system described in this disclosure is obtained when using an adsorption purifying unit with a fast cycle time, as illustrated in FIG. 8, since the size of the holding tank may be reduced to a volume being less than 2% (preferably less than 1% or even more preferably less than 0.5%) of the total volume produced by the bioreactor per 24 hours.

The present disclosure relates to a method for purifying a feed comprising a concentration of at least one target product in a chromatography system having at least a first adsorption purifying unit, the at least first adsorption purifying unit has a capacity for binding the target product and is configured to receive the feed from a first holding tank and is configured to provide the at least one target product at an outlet. The first holding tank is configured to receive continuous feed from a bioreactor, wherein the method comprising:
a) loading the at least first adsorption purifying unit with a volume of feed provided from the first holding tank, the volume of feed comprising an amount of the at least one target product corresponding to less than, or equal to, the capacity for binding the target product in the at least first adsorption purifying unit;
b) when step a) is completed, washing, eluting, cleaning and regenerating the at least first adsorption purifying unit while filling the first holding tank with feed, said first holding tank having a volume of at least the amount of the feed provided by the bioreactor during this step;
c) repeating step a) and b) for a predetermined number of cycles.

The capacity for binding the target product in the adsorption purifying unit is related to the concentration of the at least one target product in the feed and the volume of feed loaded into the adsorption purifying unit.

According to some aspects, the first holding tank has a maximum volume and contains a fraction of the total volume when step a) is completed. The fraction of the total volume is larger than zero, but may be any portion of the maximum volume, such as ¾, ¼ of the maximum volume. According to some aspects, the holding tank is empty, or very close to empty, after loading (step a). However, this introduces a risk of sucking air into the purifying unit at the end of the loading step.

According to some aspects, the chromatography system further comprises a weight sensor configured to measure a weight, W, of the first holding tank, wherein step a) and step b) are initiated based on the measured weight of the first holding tank. The measured weight, W, is a measure of the amount of target product available in the holding tank when the concentration of the target product in the feed is known.

According to some aspects, the method further comprises initiating step a) when the measured weight of the first holding tank is above a first weight threshold, W>W1; and initiating step b) when the measured weight of the first holding tank is below a second weight threshold, W<W2.

According to some aspects, step a) is performed during a time period, T, while filling the first holding tank with feed.

According to some aspects, step a) further comprises: a1) determining a target product concentration, C_{TP} in the first holding tank using a sensor; and a2) setting the time period, T, based on the target product concentration in the first holding tank.

According to some aspects, the step of determining the target product concentration, C_{TP}, is performed by measuring the concentration in the first holding tank. This may be done using a sensor based on Surface Plasmon Resonance, SPR, in the first holding tank.

According to some aspects, the at least first adsorption purifying unit is a single adsorption purifying unit or multiple parallel-connected adsorption purifying units.

According to some embodiments, the loading in step a) is performed at a loading flow rate, R_{L}, and the feed from the bioreactor is introduced into the first holding tank at a feed flow rate, R_{F}, wherein the feed flow rate is lower than the loading flow rate, R_{F}<R_{L}.

Bioprocesses utilizing bioreactors with continuous feed output, such as many perfusion processes, are typically run for long durations, in general from around 10 days up to several months. As long processing times increase process risk in terms of bioburden introduction, it is desired to run these processes in closed systems to avoid opening up the process to contamination. Therefore, it is in most cases beneficial to use the same adsorption purifying unit(s) throughout the entire process duration, eliminating the need to open up the process for replacing purifying units. Considering this, adding the fact that adsorption purifying units typically have a lifetime of up to at best around 200 cycles, it is easily realized that the typical duration of a chromatography cycle should be designed to last for a duration of around one hour up to around 10 hours, depending on setup (Equation I).

$$\frac{Bioreactor production duration ( days ) \times 24 ( h ) \times 60 ( min )}{Adsorption purifying unit lifetime ( \# cycles )} = \qquad Equation I$$

$$Cycle time ( min ) \\ Cycle time calculation$$

In contrast to the long cycle time duration, all the non-loading steps such as washing, eluting, cleaning, CIP, and regenerating the column(s) before it may be loaded with feed again should still be designed to run rapidly in order to reduce the time product is not loading and thereby keeping holding tank volume requirements low. Non-loading steps may typically be completed in less than 15 minutes in the case of membrane chromatography and less than 30 minutes in the case of optimized resin-based chromatography. In these cases, as the cycle time consists of loading time and time for non-loading steps (Equation II), the loading will be run at a low flow rate to ensure total cycle time criteria are met. This means that the loading time will make up the majority of the total cycle time.

Total cycle time=Loading time+Washing time+Elution time+Cleaning time+CIP time+Regeneration time=Loading time+Non-loading time Equation II: Total cycle time

As a consequence of the long loading times, the loading flow rate will typically be similar to the flow rate of perfusate coming out from the bioreactor. For example, in case of 10 days perfusion production, 100 cycles adsorption purifying unit lifetime and 30 min non-loading time, the cycle time will be 144 minutes, loading time will be 114 minutes and the loading flow rate will be 26% higher than the perfusate flow rate (Equation III).

$$\frac{Loading flow rate ( L / min )}{Bioreactor perfusate flow rate ( L / min )} = \qquad Equation III$$

$$\frac{Total cycle time ( min )}{Total cycle time ( min ) - Non loading cycle time ( min )} \\ Loading flow rate vs Bioreactor perfusate flow rate calculation$$

At longer process durations and shorter loading times, the difference between loading flow rate and perfusate flow rate is even smaller. For example, if the perfusion production duration is 30 days and non-loading time is 10 min, while purifying unit lifetime remains 100 cycles, the cycle time will be 432 minutes, loading time will be 422 minutes and the loading flow rate will be only 2.4% higher than the perfusate flow rate.

According to some aspects, the method further comprising:
d) redirecting the feed from the bioreactor to at least a second adsorption purifying unit after repeating steps a)-c) using the at least first adsorption purifying unit the predetermined number of cycles; and
e) repeating steps a)-c) while providing feed from the bioreactor to the at least second adsorption purifying unit.

According to some aspects, the chromatography system is further provided with a first valve having an inlet for receiving feed from the bioreactor, a first outlet connected to the first holding tank and a second outlet connected to a second holding tank which is configured to provide feed to the at least second adsorption purifying unit, wherein step d) further comprises:
d1) redirecting the feed flow from the bioreactor to the second holding tank using the first valve after the predetermined number of cycles.

According to some aspects, the chromatography is further provided with a second valve having at least one inlet for receiving feed from the first holding tank, a first outlet connected to the at least first adsorption purifying unit and a second outlet connected to the at least second adsorption purifying unit, wherein step d) further comprises:
d2) redirecting the feed flow from the bioreactor to the at least second adsorption purifying unit using the second valve after the predetermined number of cycles.

According to some aspects, the method further comprising:
e1) redirecting the feed from the bioreactor from the first holding tank to a second holding tank; and
e2) loading the first adsorption purifying unit with feed from the second holding tank.

According to some aspects, the predetermined number of cycles is based on the performance of the at least first adsorption purifying unit. The repeating of steps a)-c) is maintained as long as the performance is maintained, and the performance may be monitored using sensors to detect the status of the adsorption purifying unit. Status is obtained by monitoring the change in certain parameters over the adsorption purifying unit, such as pressure difference (dP), or comparing parameters before and after the purifying unit, such as UV, pH, conductivity, etc. Also, it is possible to evaluate the UV elution peak area and peak shape or salt transition shapes.

The present disclosure also relates to a chromatography system for purifying a feed comprising a concentration of at least one target product. The chromatography system has at least a first adsorption purifying unit, and the at least first adsorption purifying unit has a capacity for binding the target product and is configured to receive the feed from at least a first holding tank. The first adsorption purifying unit is also configured to provide the at least one target product at an outlet, and the at least first holding tank is configured to receive continuous feed from a bioreactor. The chromatography system further comprises a control unit configured to perform the steps:
a) loading the at least first adsorption purifying unit with a volume of feed provided from the first holding tank, the volume of feed comprising an amount of the at least one target product corresponding to less than, or equal to, the capacity for binding the target product in the at least first adsorption purifying unit;
b) when step a) is completed, washing, eluting, cleaning and regenerating the at least first adsorption purifying unit while filling the first holding tank with feed, said first holding tank having a volume of at least the amount of the feed provided by the bioreactor during this step;
c) repeating step a) and b) for a predetermined number of cycles.

The present disclosure also relates to a computer program for controlling process parameters in a chromatography system, comprising instructions which, when executed on at least one processor, cause the at least one processor to carry out the method according to any of the steps presented above.

The present disclosure also relates to a computer-readable storage medium carrying a computer program for controlling process parameters in a chromatography system.

Aspects of the disclosure are described with reference to the drawings, e.g., block diagrams and/or flowcharts. It is understood that several entities in the drawings, e.g., blocks of the block diagrams, and also combinations of entities in the drawings, can be implemented by computer program instructions, which instructions can be stored in a computer-readable memory, and also loaded onto a computer or other programmable data processing apparatus. Such computer program instructions can be provided to a processor of a general purpose computer, a special purpose computer and/or other programmable data processing apparatus to produce a machine, such that the instructions, which execute via the processor of the computer and/or other programmable data processing apparatus, create means for implementing the functions/acts specified in the block diagrams and/or flowchart block or blocks.

In some implementations and according to some aspects of the disclosure, the functions or steps noted in the blocks can occur out of the order noted in the operational illustrations. For example, two blocks shown in succession can in fact be executed substantially concurrently or the blocks can sometimes be executed in the reverse order, depending upon the functionality/acts involved. Also, the functions or steps noted in the blocks can according to some aspects of the disclosure be executed continuously in a loop.

In the drawings and specification, there have been disclosed exemplary aspects of the disclosure. However, many variations and modifications can be made to these aspects without substantially departing from the principles of the present disclosure. Thus, the disclosure should be regarded as illustrative rather than restrictive, and not as being limited to the particular aspects discussed above. Accordingly, although specific terms are employed, they are used in a generic and descriptive sense only and not for purposes of limitation.

The description of the example embodiments provided herein have been presented for purposes of illustration. The description is not intended to be exhaustive or to limit example embodiments to the precise form disclosed, and modifications and variations are possible in light of the above teachings or may be acquired from practice of various alternatives to the provided embodiments. The examples discussed herein were chosen and described in order to explain the principles and the nature of various example embodiments and its practical application to enable one skilled in the art to utilize the example embodiments in various manners and with various modifications as are suited to the particular use contemplated. The features of the embodiments described herein may be combined in all possible combinations of methods, apparatus, modules, systems, and computer program products. It should be appreciated that the example embodiments presented herein may be practiced in any combination with each other.

It should be noted that the word “comprising” does not necessarily exclude the presence of other elements or steps than those listed and the words “a” or “an” preceding an element do not exclude the presence of a plurality of such elements. It should further be noted that any reference signs do not limit the scope of the claims, that the example embodiments may be implemented at least in part by means of both hardware and software, and that several “means”, “units” or “devices” may be represented by the same item of hardware.

The various example embodiments described herein are described in the general context of method steps or processes, which may be implemented in one aspect by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

In the drawings and specification, there have been disclosed exemplary embodiments. However, many variations and modifications can be made to these embodiments. Accordingly, although specific terms are employed, they are used in a generic and descriptive sense only and not for purposes of limitation, the scope of the embodiments being defined by the following claims.

1. A method for purifying a feed comprising a concentration of at least one target product in a chromatography system having at least a first adsorption purifying unit, the at least first adsorption purifying unit has a capacity for binding the target product and is configured to receive the feed from a first holding tank and to provide the at least one target product at an outlet, wherein the first holding tank is configured to receive a continuous feed from a bioreactor, the method comprising:
a) loading the at least first adsorption purifying unit with a volume of feed provided from the first holding tank, the volume of feed comprising an amount of the at least one target product corresponding to less than, or equal to, the capacity for binding the target product in the at least first adsorption purifying unit;
b) when step a) is completed, washing, eluting, cleaning and regenerating the at least first adsorption purifying unit while filling the first holding tank with feed, said first holding tank having a volume of at least the amount of the feed provided by the bioreactor during this step; and
c) repeating step a) and b) for a predetermined number of cycles.

2. The method according to claim 1, wherein the first holding tank has a maximum volume and contains a fraction of the total volume when step a) is completed.

3. The method according to claim 1, wherein the chromatography system further comprises a weight sensor configured to measure a weight, W, of the first holding tank, wherein step a) and step b) are initiated based on the measured weight of the first holding tank.

4. The method according to claim 3, wherein the method further comprises:
initiating step a) when the measured weight of the first holding tank is above a first weight threshold, W>W1; and
initiating step b) when the measured weight of the first holding tank is below a second weight threshold, W<W2.

5. The method according to claim 1, wherein step a) is performed during a time period, T, while filling the first holding tank with feed.

6. The method according to claim 5, wherein the step of determining the target product concentration, C_{TP}, is performed by measuring the concentration in the first holding tank.

7. The method according to claim 1, wherein the at least first adsorption purifying unit is a single adsorption purifying unit or multiple parallel-connected adsorption purifying units.

8. The method according to claim 1, wherein the loading in step a) is performed at a loading flow rate, R_{L}, and the feed from the bioreactor is introduced into the first holding tank at a feed flow rate, R_{F}, wherein the feed flow rate is lower than the loading flow rate, R_{F}<R_{L}.

9. The method according to claim 1, further comprising:
d) redirecting the feed from the bioreactor to at least a second adsorption purifying unit after repeating steps a)-c) using the at least first adsorption purifying unit the predetermined number of cycles; and
e) repeating steps a)-c) while providing feed from the bioreactor to the at least second adsorption purifying unit.

10. The method according to claim 9, wherein the chromatography system is further provided with a first valve having an inlet for receiving feed from the bioreactor, a first outlet connected to the first holding tank and a second outlet connected to a second holding tank which is configured to provide feed to the at least second adsorption purifying unit, wherein step d) further comprises:
d1) redirecting the feed flow from the bioreactor to the second holding tank using the first valve after the predetermined number of cycles.

11. The method according to claim 9, wherein the chromatography is further provided with a second valve having at least one inlet for receiving feed from the first holding tank, a first outlet connected to the at least first adsorption purifying unit and a second outlet connected to the at least second adsorption purifying unit, wherein step d) further comprises:
d2) redirecting the feed flow from the bioreactor to the at least second adsorption purifying unit using the second valve after the predetermined number of cycles.

12. The method according to claim 1, further comprising:
e1) redirecting the feed from the bioreactor from the first holding tank to a second holding tank; and
e2) loading the first adsorption purifying unit with feed from the second holding tank.

13. The method according to claim 1, wherein the predetermined number of cycles is based on the performance of the at least first adsorption purifying unit.

14. A chromatography system for purifying a feed comprising a concentration of at least one target product, the chromatography system having at least a first adsorption purifying unit, the at least first adsorption purifying unit has a capacity for binding the target product and is configured to receive the feed from at least a first holding tank and to provide the at least one target product at an outlet, wherein the at least first holding tank is configured to receive continuous feed from a bioreactor, the chromatography system further comprises a control unit configured to perform the steps according to claim 1.

5. A computer program for controlling process parameters in a chromatography system, comprising instructions which, when executed on at least one processor, cause the at least one processor to carry out the method according to claim 1.

16. A computer-readable storage medium carrying a computer program for controlling process parameters in a chromatography system according to claim 15.
